(19) United States
(12) Patent Application Publication
(10) Pub. No.: US 2006/0240128 A1
(43) Pub. Date: Oct. 26, 2006

(54) Combined immediate release and extended release analgesic composition

(76) Inventor: Thomas G. Schlagheck, Naples, FL (US)

Correspondence Address: DILWORTH & BARRESE, LLP, 333 EARLE OVINGTON BLVD., UNIONDALE, NY, 11553, US

(21) Appl. No.: 10/527,077

(22) PCT Filed: Sep. 8, 2003
(86) PCT No.: PCT/US03/28042

§ 371 Date: Jun. 28, 2006

Related U.S. Application Data
(60) Provisional application No. 60/409,154, filed on Sep. 9, 2002.

(51) Int. Cl. A61K 36/81; A61K 31/485; A61K 31/445; A61K 31/13; A61K 31/137

(52) U.S. Cl. 424/725.1; 424/760; 514/649; 514/663; 514/282; 514/317

(57) ABSTRACT

The present invention pertains to an analgesic composition comprising an analgesic drug in an extended release form in combination with an analgesia-enhancing amount of a nontoxic N-methyl-D-aspartate receptor antagonist in an immediate release form.

### CROSS-REFERENCE TO RELATED APPLICATIONS

This application claims the benefit under 35 U.S.C. §119(e) of earlier filed and copending U.S. Provisional Application No. 60/409,154, filed Sep. 9, 2002, the contents of which are incorporated herein by reference.

### BACKGROUND OF THE INVENTION

1. Field of the Invention

The present invention relates to a combined analgesic composition having at least one analgesic drug in an extended release form and at least one nontoxic N-methyl-D-aspartate receptor antagonist in an immediate release form, where the activity of the analgesic drug is enhanced by the at least one nontoxic N-methyl-D-aspartate receptor antagonist. Preferably, the analgesic drug is an opioid analgesic, the at least one nontoxic N-methyl-D-aspartate receptor antagonist is dextromethorphan, and the analgesic composition is substantially free of opioid antagonist.

2. Description of the Related Art

Analgesics are a class of pharmaceutical compounds known for their ability to reduce the perception of pain. Known analgesics include, but are not limited to, opioid analgesics, non-narcotic analgesics, coal tar analgesics, nonsteroidal anti-inflammatory drugs (NSAIDs), gabapentin, substance P antagonists, capsaicin or capsaicinoids, and cyclooxygenase-II (COX II) inhibitors.

Morphine, a classic opioid, has been known as a very powerful analgesic compound for many years. Its potential as a target of abuse has been known for almost as long. Opioids and their derivatives are used in the pharmaceutical industry as narcotic analgesics, hypnotics, sedatives, anti-diarrheals, anti-spasmotics, and antitussives. Despite their well known potential for addiction and abuse, opioids are widely used due to their superior, powerful analgesic properties.

In the past, abuse of opioids was generally limited to illicit drugs made in illegal laboratories. Abuse of pharmaceutical opioids was quite limited. Accordingly, action by makers of pharmaceutical opioids would, in the past, have little or no effect on illegal abuse of opioids.

Recently, however, this trend has been changing and abuse of pharmaceutical opioids has been increasing. This is especially true in the case of extended release opioid dosage forms. One reason for the increase of abuse is that extended release opioid dosage forms are intended for decreased frequency of dosing, which results in the production of dosage forms having substantially increased amounts of opioid. Therefore, an extended release dosage form can provide much more opioid to the potential abuser than the past low dose, immediate release dosage forms.

N-methyl-D-aspartate (NMDA) receptor antagonists are well known in the art and encompass, for example, dextromethorphan, dextrorphan, memantine, amantidine, d-methadone and their pharmaceutically acceptable salts. NMDA receptor antagonists are known to inhibit the development of tolerance to and/or dependence on addictive drugs, e.g., narcotic analgesics such as morphine, codeine, etc., as disclosed in U.S. Pat. Nos. 5,321,012 and 5,556,838, and to treat chronic pain as disclosed in U.S. Pat. No. 5,502,058, the contents of each of which are incorporated by reference herein.

Nontoxic NMDA receptor antagonists, such as dextromethorphan, are also known to enhance the effects of some drugs, especially opioid analgesics. See, e.g., U.S. Pat. Nos. 5,502,058 and 5,840,731, respectively, the contents of which are incorporated by reference herein. In some cases, the nontoxic NMDA receptor antagonist is administered in combination with a local anesthetic. See U.S. Pat. No. 5,352,683, the contents of which are incorporated by reference herein.

Excessive levels of nontoxic NMDA receptor antagonists are to be avoided, however, as they can present adverse side effects similar to those caused by opioids, which include, but are not limited to, constipation, nausea, headache, vomiting, itchiness, dizziness, sleepiness, drowsiness, weakness, fatigue, confusion and/or disorientation.

Two examples of previous attempts to curtail abuse of opioids, U.S. Pat. Nos. 6,228,863 and 6,277,384, both disclose single unit dosage forms containing an opioid, an opioid antagonist and, optionally, any of a third group of drugs among which are mentioned NMDA receptor antagonists. The opioid antagonist counteracts the euphoric effects of the opioid and renders the dosage form less likely to be abused.

Controlled release dosage forms for pharmaceuticals, which include extended release and sustained release dosage forms, are known to those skilled in the art. See, e.g., U.S. Pat. Nos. 4,861,598, 4,970,075, 5,266,331, 5,508,042, 5,549,912, 5,656,295, 5,958,459, 5,968,551, 6,103,261, 6,143,322, 6,143,353, and 6,294,195, the contents of each of which are incorporated by reference herein. For example, U.S. Pat. Nos. 4,861,598 and 4,970,075 disclose controlled release pharmaceutical compositions for oral administration having extended action due to their use of a higher aliphatic alcohol and acrylic resin as their base material. Pharmaceutically active agents utilized with these compositions include narcotics and NMDA receptor antagonists. U.S. Pat. Nos. 5,266,331, 5,508,042, 5,549,912 and 5,656,295 disclose solid controlled release oral dosage forms of oxycodone or its salts whereby the oxycodone is encompassed in a carrier with a defined dissolution rate for the extended release of the pharmaceutical in vitro. U.S. Pat. No. 6,194,000 discloses pharmaceutical compositions which include an NMDA receptor antagonist in a controlled release form.

It would be beneficial to develop an analgesic composition which contains an analgesic drug in conjunction with a nontoxic NMDA receptor antagonist whereby the nontoxic NMDA receptor antagonist is present in an amount which enhances the effects of the analgesic drug, thereby reducing the amount of analgesic necessary to obtain the same effect, but the nontoxic NMDA receptor antagonist is not present in such an amount as to present adverse side effects.

### BRIEF SUMMARY OF THE INVENTION

The present invention relates to an analgesic composition comprising at least one analgesic drug in an extended release form in combination with an analgesia-enhancing amount of at least one nontoxic N-methyl-D-aspartate antagonist in an immediate release form. Because of the analgesia-enhancing effects of the nontoxic N-methyl-D-aspartate antagonist, lower doses of the analgesic drug may be used to obtain the same effect.

In addition, by having the nontoxic N-methyl-D-aspartate receptor antagonist available for immediate release, the analgesic composition can utilize lower amounts of nontoxic N-methyl-D-aspartate receptor antagonist to achieve the same analgesic effect than if the nontoxic N-methyl-D-aspartate receptor antagonist was in an extended release form. This reduces the potential for negative or adverse side effects and optimizes the analgesia-enhancing effects of the nontoxic N-methyl-D-aspartate receptor antagonist in the composition of the present invention. Preferably, the weight ratio of the analgesic drug to the nontoxic NMDA receptor antagonist is 2:1. In a preferred embodiment the analgesic drug in extended release form is an opioid analgesic, the nontoxic N-methyl-D-aspartate antagonist in immediate release form is dextromethorphan, and the analgesic composition is substantially free of opioid antagonist.

### DETAILED DESCRIPTION OF THE INVENTION

The present invention is directed to analgesic compositions comprising a combination of at least one analgesic drug in an extended release form and at least one nontoxic NMDA receptor antagonist in an immediate release form. The nontoxic NMDA receptor antagonist is present in an amount which enhances the analgesia of the analgesic drug. Preferably, the analgesic drug is an opioid analgesic and the analgesic composition is substantially free of opioid antagonist.

The first component of the analgesic composition is an analgesic drug in an extended release form. The analgesic drug is a pharmacologically active substance e.g., a pharmaceutically useful amount of an opioid analgesic, a non-narcotic analgesic such as acetaminophen, a nonsteroidal anti-inflammatory drug (NSAID) such as aspirin, bromfenac, diclofenac, diflusinal, etodolac, fenbufen, fenoprofen, flufenisal, flurbiprofen, ibuprofen, indomethacin, ketoprofen, ketorolac, meclofenamic acid, mefenamic acid, nabumetone, naproxen, oxaprozin, phenylbutazone, piroxicam, sulindac, tolmetin, zomepirac, and the like, gabapentin, substance P antagonists, capsaicin or capsaicinoids, cyclooxygenase-II (COX II) inhibitors such as celecoxib (Celebrex), rofecoxib (Vioxx), meloxicam, L-745337 (Merck), MK-966 (Merck), L-768277 (Merck), GR-253035 (Glaxo-Wellcome), JTE-S22 (Japan Tobacco), RS-57067-000 (Roche), SC-58125 (Searle), SC-078 (Searle), PD-138387 (Warner-Lambert), NS-398 (Taisho), flosulide, valdecoxib (Bextra), lumiracoxib (Prexige), etoricoxib (Arcoxia), DUP-697 (Dupont), celebra (Pfizer), parecoxib (Pharmacia) and PD-164387 (Warner-Lambert). These and other COX-II inhibitors are described in, e.g., U.S. Pat. Nos. 6,239,173; 6,063,811; 5,691,374; 5,474,995; 5,972,986; 5,760,068; 5,563,165; 5,466,823; 5,616,601; 5,604,260; 5,593,994; 5,550,142; 5,536,752; 5,521,213; 5,639,780; 5,604,253; 5,552,422; 5,510,368; 5,436,265; 5,409,944; and 5,130,311, all of which are hereby incorporated by reference.

Preferably, the analgesic drug is an opioid analgesic present in an analgesically effective amount and the analgesic composition is substantially free of opioid antagonist. Opioid analgesics suitable for use in the analgesic composition generally have a potential for abuse and include, but are not limited to, alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphine, normorphine, norpipanone, opium, oxycodone, oxymorphone, papaveretum, pentazocine, phenadoxone, phenomorphan, phenazocine, phenoperidine, piminodine, piritramide, propheptazine, promedol, properidine, propoxyphene, sufentanyl, tilidine, tramadol and their pharmaceutically acceptable salts.

Where the first component of the analgesic composition is an opioid analgesic, opioid antagonists, including but are not limited to naltrexone, naloxone, cyclazocine, levallorphan, and their pharmaceutically acceptable salts, are substantially excluded from the analgesic composition since they pose a risk of precipitating opioid withdrawal when taken by a chronic opioid abuser.

The preferred daily dosage of opioid analgesic can range from about 1 mg per 70 kg body weight to about 800 mg per 70 kg body weight, depending on the opioid used. Preferably, the daily dosage of opioid analgesic is from about 10 mg per 70 kg body weight to about 500 mg per 70 kg body weight. Where the opioid analgesic is fentanyl or sufentanyl, the daily dosage can range from about 100 μg per 70 kg to about 6 mg per 70 kg body weight, and preferably from about 250 μg to about 3 mg per 70 kg body weight. Due to their potency, rapid metabolization and highly undesirable side effects following overdosage (most notably respiratory depression, which if left unchecked can cause death), fentanyl and its even more potent derivative sufentanyl are preferably administered topically for transdermal delivery by diffusion through the epidermis.

The second component of the analgesic composition is at least one nontoxic NMDA receptor antagonist. The nontoxic NMDA receptor antagonist is present in the analgesic composition in an immediate release form, e.g., by being present in the analgesic composition in an unmodified state capable of immediate absorption, by being contained in an immediate release carrier, by being applied to the exterior surface of the extended release form containing the analgesic drug, or by being contained in the coating of the extended release form.

Nontoxic NMDA receptor antagonists suitable for use in accordance with the present invention include dextromethorphan ((+)-3-hydroxy-N-methylmorphinan), its metabolite dextrorphan ((+)-3-hydroxy-N-methylmorphinan), amantadine (1-amino adamantine), memantine (3,5 dimethylaminoadamantone), d-methadone (d-form of 6-dimethylamino-4,4-diphenyl-3-heptanone hydrochloride), their mixtures and their pharmaceutically acceptable salts. Dextromethorphan is a preferred NMDA receptor antagonist for use herein due to its ready availability and wide acceptance as an ingredient of many over-the-counter medications where it is utilized for its cough-suppressant (antitussive) activity.

The term “nontoxic” as used herein shall be understood in a relative sense and is intended to designate any substance that has been approved by the United States Food and Drug Administration (“FDA”) for administration to humans or, in keeping with established regulatory criteria and practice, is susceptible to approval by the FDA for administration to humans. The term “nontoxic” is also used herein to distinguish the NMDA receptor antagonists that are useful in the practice of the present invention from NMDA receptor antagonists such as MK 801 (the compound 5-methyl-10,11-dihydro-SH-dibenze[a,d]cyclohepten-5,10-imine), CPP (the compound 3-[2-carboxypiperazin-4-yl]propyl-1-phosphonic acid) and PCP (the compound 1-(1-phenylcyclohexyl)piperidine) whose toxicities effectively preclude their therapeutic use.

The nontoxic NMDA receptor antagonist is present in an amount which enhances the pharmacological effects of the analgesic drug. As used herein, the terms “enhance”, “enhances”, “enhancing”, “analgesia-enhancing amount”, and “enhancement” maybe used interchangeably and are understood to mean an amount of nontoxic NMDA receptor antagonist which does one of the following: (i) increases levels of analgesia so that analgesia resulting from the analgesic composition of the present invention is greater than the sum of the analgesic effects attributable to the analgesic drug and nontoxic NMDA receptor antagonist components when each of these components is administered alone, (ii) provides the same level of analgesia using a lower amount of analgesic compared to the analgesic alone, (iii) creates a synergistic effect when administered with the analgesic so that analgesia is obtained when the analgesic composition of the present invention is administered, but would not be obtained if the nontoxic NMDA receptor antagonist and analgesic were administered alone and to the exclusion of the other; (iv) suppresses or minimizes any adverse effects of the analgesic drug.

Where the first drug is an opioid analgesic, the nontoxic NMDA receptor antagonist is present in an opioid analgesia-enhancing amount. For purposes of this disclosure, an “opioid analgesia-enhancing amount” of nontoxic NMDA receptor antagonist is one which does one of the following: (i) increases levels of analgesia compared with the administration of an opioid analgesic alone, (ii) provides the same level of analgesia using a lower amount of opioid compared to the opioid alone, (iii) delays the onset of dependency to the opioid analgesic, or (iv) delays the onset of tolerance to the opioid analgesic.

For purposes of this disclosure, “extended release” includes “controlled release” and “sustained release” and pertains to the release of pharmaceutical agents at a defined level over an extended period of time.

The expression “dosage form” is understood to include “unit dosage form”. The expression “unit dosage form” means a physically discrete unit which contains specified amounts of the analgesic drug in an extended release form in combination with the nontoxic NMDA receptor antagonist in immediate release form, and any other pharmacologically active substance or pharmaceutical excipient, which amounts are selected so that a fixed number, e.g. one, of the units is suitable to achieve a desired therapeutic effect.

All modes of administration are contemplated, e.g., orally, rectally, parenterally, intrathecally, intranasally, transdermally, and topically.

The preferred daily dosage of nontoxic NMDA receptor antagonist can range from about 10 mg per 70 kg body weight to about 750 mg per 70 kg body weight. Preferably, the daily dosage of nontoxic NMDA receptor antagonist is from about 30 mg per 70 kg body weight to about 500 mg per 70 kg body weight. In a most preferred embodiment the nontoxic NMDA receptor antagonist is dextromethorphan.

It is also within the scope of this invention to include with the nontoxic NMDA receptor antagonist a local anesthetic such as bupivicaine hydrochloride, chloroprocaine hydrochloride, dibucaine, dibucaine hydrochloride, etidocaine hydrochloride, lidocaine, lidocaine hydrochloride, mepivacaine hydrochloride, piperocaine hydrochloride, prilocaine hydrochloride, procaine hydrochloride, propoxycaine hydrochloride, tetracaine, tetracaine hydrochloride, and the like.

The nontoxic NMDA receptor antagonist must be present in the analgesic composition in an analgesia-enhancing amount. It would be recognized by one skilled in the art that this amount will relate to the nature and amount of the analgesic drug present and its analgesia-inducing capacity, the nature of the nontoxic NMDA receptor antagonist and its ability to enhance the analgesia effect, as well as the particular formulation containing the active substances. As those skilled in the art will recognize, many factors that modify the action of the active substances herein, such as the state and circumstances of the host being treated, will be taken into account by the treating physician such as the age, body weight, sex, diet and condition of the subject, including metabolic status, the time of administration, the rate and route of administration, and so forth. Optimal dosages for a given set of conditions can be ascertained by those skilled in the art using conventional dosage determination tests.

The ratio of nontoxic NMDA receptor antagonist, such as dextromethorphan, to the analgesic drug is important in providing the optimal analgesic effect. In general, a weight ratio of analgesic drug in extended release form to nontoxic NMDA receptor antagonist in immediate release form can range from about 2:1 to about 1:10, and preferably from about 1:1 to about 1:5 by weight.

For example, where the analgesic drug is an opioid analgesic, such as morphine, a 1:1 ratio of morphine to dextromethorphan in immediate release formulations has been shown to enhance the effect of morphine alone. Further increasing the ratio of morphine to dextromethorphan to 1:2 increases the enhancement effect, but dextromethorphan associated adverse events may limit rising doses of dextromethorphan. However, in accordance with the present invention, a higher ratio of dextromethorphan to opioid analgesic may be obtained systemically with lower amounts of dextromethorphan, if 100% of the dextromethorphan is immediately released while a portion of the opioid analgesic is released over time. The release of 100% dextiomethorphan as an immediate release component (IR) provides greater amounts of dextromethorphan to morphine in an extended release component (ER) on an absolute μmolar basis at the systemic level, compared to where both drugs are administered as extended release components (ER-ER) as per the following table:

TABLE 1

| Formulation | Analgesic/ | Absolute analgesic: |
|---|---|---|
| Analgesic/ | dextromethorphan | dextromethorphan ratio at |
| dextromethorphan | (mg/mg) | release & over time |
| ER - ER | 60/60 | 1:1 |
| ER - IR | 60/60 | 1:2* |
| ER - IR | 60/30 | 1:1 |

*assumes ≈50% of the ER analgesic is released during initial dissolution; effective ratio at the cellular level may be higher with subsequent release of analgesic over time.

As is apparent from the above table, there is a 2-fold or more increase in absolute ratio of analgesic to dextromethorphan at the systemic level when equimolar amounts of dextromethorphan IR are administered compared with dextromethorphan ER. Thus, 50% less dextromethorphan IR will achieve, in this example, a minimum 1:1 ratio of dextromethorphan to the analgesic at the systemic level. The lower amount of dextromethorphan required to provide the needed ratio of dextromethorphan to analgesic will minimize or reduce any adverse side effects attributed to dextromethorphan when the analgesic composition of the present invention is administered to a patient.

While not wishing to be bound by any theory, loading NMDA receptors with dextromethorphan soon after drug administration and then metering out the analgesic drug, such as an opioid analgesic, may pharmacologically optimize the enhancing effects of the nontoxic NMDA receptor antagonist on the analgesic drug.

Additionally, the analgesic composition herein can optionally contain at least one other pharmacologically active substance e.g., a pharmaceutically useful amount of an analgesic drug as described above, including a non-narcotic analgesic such as acetaminophen, a nonsteroidal anti-inflammatory drug (NSAID) such as aspirin, bromfenac, diclofenac, diflusinal, etodolac, fenbufen, fenoprofen, flufenisal, flurbiprofen, ibuprofen, indomethacin, ketoprofen, ketorolac, meclofenamic acid, mefenamic acid, nabumetone, naproxen, oxaprozin, phenylbutazone, piroxicam, sulindac, tolmetin, zomepirac, and the like, gabapentin, substance P antagonists, capsaicin or capsaicinoids, cyclooxygenase-II (COX II) inhibitors, or anesthetics.

The analgesic compositions provide an extended release of the analgesic drug and an immediate release of the NMDA receptor antagonist. Such embodiments may further comprise a portion of the analgesic drug in immediate release form. Sustained release of the analgesic drug may be accomplished in accordance with formulations/methods of manufacture known to those skilled in the art of pharmaceutical formulation, e.g., via the incorporation of the analgesic drug in an extended release carrier; or via a controlled release coating of a carrier containing the analgesic drug.

In one embodiment, the analgesic composition comprises at least one analgesic drug in an extended release form in combination with at least one nontoxic NMDA receptor antagonist in an unmodified state capable of immediate release. In another embodiment, the sustained release carrier containing the analgesic drug is combined with an immediate release carrier containing the nontoxic NMDA receptor antagonist. The nontoxic NMDA receptor antagonist may also be applied to the exterior surface of the extended release carrier and is thus available for immediate release. Alternatively, the analgesic drug may be contained in a normal release carrier having a coating that controls the release of the drug. In such a case, the coating may contain the nontoxic NMDA receptor antagonist, which is available for immediate release.

Suitable base materials for controlled release carriers include combinations of higher aliphatic alcohols and acrylic resins. Base compositions prepared from such higher aliphatic alcohols and acrylic resins provide sustained release of therapeutically active ingredients over a period of time from five hours and for as much as 24 hours after administration, generally oral administration, in humans or animals.

These bases can be prepared from any pharmaceutically acceptable higher aliphatic alcohol, the most preferred being fatty alcohols of 10-18 carbon atoms, particularly stearyl alcohol, cetyl alcohol, cetostearyl alcohol, lauryl alcohol, myristyl alcohol and mixtures thereof.

Any acrylic polymer which is pharmaceutically acceptable can be used for the purposes of the present invention. The acrylic polymers may be cationic, anionic or non-ionic polymers and may be acrylates, methacrylates, formed of methacrylic acid or methacrylic acid esters. These polymers can be synthesized, as indicated above, to be cationic, anionic or non-ionic, which then renders the polymers that would be pH dependent and consequently soluble in, or resistant to solutions over a wide range in pH.

In addition, suitable materials for inclusion in a controlled release carrier include:

(a) Hydrophilic polymers, such as gums, cellulose ethers, acrylic resins and protein derived materials. Of these polymers, the cellulose ethers, especially hydroxyalkylcelluloses and carboxyalkylcelluloses, are preferred. The analgesic composition may contain between 1% and 80% (by weight) of at least one hydrophilic or hydrophobic polymer.

(b) Digestible, long chain (C₈-C₅₀, especially C₁₂-C₄₀), substituted or unsubstituted hydrocarbons, such as fatty acids, fatty alcohols, glyceryl esters of fatty acids, mineral and vegetable oils and waxes. Hydrocarbons having a melting point of between 25° and 90° C. are preferred. Of these long chain hydrocarbon materials, fatty (aliphatic) alcohols are preferred. The oral dosage form may contain up to 60% (by weight) of at least one digestible, long chain hydrocarbon.

(c) Polyalkylene glycols. The oral dosage form may contain up to 60% (by weight) of at least one polyalkylene glycol.

One particularly suitable carrier comprises at least one water soluble hydroxyalkyl cellulose, at least one C₁₂-C₃₆, preferably C₁₄-C₂₂, aliphatic alcohol and, optionally, at least one polyalkylene glycol.

The at least one hydroxyalkyl cellulose is preferably a hydroxy (C₁ to C₆) alkyl cellulose, such as hydroxypropylcellulose, hydroxypropylmethylcellulose and, especially, hydroxyethyl cellulose. The amount of the at least one hydroxyalkyl cellulose in the present analgesic composition will be determined, inter alia, by the precise rate of analgesic drug release required. Preferably however, the oral dosage form contains between 1% and 45%, especially between 5% and 25% (by weight) of the at least one hydroxyalkyl cellulose.

While the at least one aliphatic alcohol may be, for example, lauryl alcohol, myristyl alcohol or stearyl alcohol, in particularly preferred embodiments the at least one aliphatic alcohol is cetyl alcohol or cetostearyl alcohol. The amount of the at least one aliphatic alcohol in the present dosage form will be determined, as above, by the precise rate of analgesic drug release required. It will also depend on whether at least one polyalkylene glycol is present in or absent from the dosage form. In the absence of at least one polyalkylene glycol, the dosage form preferably contains between 20% and 50% (by weight) of the at least one aliphatic alcohol. When at least one polyalkylene glycol is present in the dosage form, then the combined weight of the at least one aliphatic alcohol and the at least one polyalkylene glycol preferably constitutes between 20% and 50% (by weight) of the total dosage.

In the present preferred dosage form, the ratio of, e.g., the at least one hydroxyalkyl cellulose or acrylic resin to the at least one aliphatic alcohol/polyalkylene glycol determines, to a considerable extent, the release rate of the analgesic drug from the formulation. A ratio of the at least one hydroxyalkyl cellulose to the at least one aliphatic alcohol/polyalkylene glycol of between 1:2 and 1:4 is preferred, with a ratio of between 1:3 and 1:4 being particularly preferred.

The at least one polyalkylene glycol may be, for example, polypropylene glycol or polyethylene glycol, which is preferred. The number average molecular weight of the at least one polyalkylene glycol is preferred between 1000 and 15000 especially between 1500 and 12000.

Another suitable controlled release carrier would comprise an alkylcellulose (especially ethyl cellulose), a C₁₂ to C₃₆ aliphatic alcohol and, optionally, a polyalkylene glycol.

In addition to the above ingredients, a controlled release carrier may also contain suitable quantities of other materials, e.g. diluents, lubricants, binders, granulating aids, colorants, flavorants and glidants that are conventional in the pharmaceutical art.

As an alternative to a controlled release carrier, the analgesic drug may be in a normal release carrier having a coating that controls the release of the drug. In particularly preferred embodiments of this aspect of the invention, the present dosage form comprises film coated spheroids containing active ingredient and a non-water soluble spheronising agent. The term spheroid is known in the pharmaceutical art and means a spherical granule having a diameter of between 0.5 mm and 2.5 mm especially between 0.5 mm and 2 mm.

The spheronising agent may be any pharmaceutically acceptable material that, together with the active ingredient, can be spheronised to form spheroids. Microcrystalline cellulose is preferred. According to a preferred aspect of the present invention, the film coated spheroids contain between 70% and 99% (by wt), especially between 80% and 95% (by wt), of the spheronising agent, especially microcrystalline cellulose.

In addition to the active ingredient and spheronising agent, the spheroids may also contain a binder. Suitable binders, such as low viscosity, water soluble polymers, will be well known to those skilled in the pharmaceutical art. However, water soluble hydroxy lower alkyl cellulose, such as hydroxy propyl cellulose, are preferred. Additionally (or alternatively) the spheroids may contain a water insoluble polymer, especially an acrylic polymer, an acrylic copolymer, such as a methacrylic acid-ethyl acrylate copolymer, or ethyl cellulose.

The spheroids are preferably film coated with a material that permits release of the analgesic drug at a controlled rate in an aqueous medium. The film coat is chosen so as to achieve, in combination with the other ingredients, the in-vitro release rate outlined above (between 12.5% and 42.5% (by weight) release after 1 hour, etc.).

The film coat will generally include a water insoluble material such as: (a) a wax, either alone or in admixture with a fatty alcohol; (b) shellac or zein; (c) a water insoluble cellulose, especially ethyl cellulose; (d) a polymethacrylate.

Preferably, the film coat comprises a mixture of the water insoluble material and a water soluble material. The ratio of water insoluble to water soluble material is determined by, amongst other factors, the release rate required and the solubility characteristics of the materials selected.

The water soluble material may be, for example, polyvinylpyrrolidone or, which is preferred, a water soluble cellulose, especially hydroxypropylmethyl cellulose.

Suitable combinations of water insoluble and water soluble materials for the film coat include shellac and polyvinylpyrrolidone or, which is preferred, ethyl cellulose and hydroxypropylmethyl cellulose. The nontoxic NMDA receptor antagonist may be applied to the exterior surface of, or included within, the film coat to provide for the immediate release of the nontoxic NMDA receptor antagonist while at the same time providing for the extended release of the analgesic drug.

In another embodiment, in order to obtain a sustained-release of the analgesic drug sufficient to provide an analgesic effect for the extended durations set forth in the present invention, the substrate comprising the therapeutically active agent may be coated with a sufficient amount of hydrophobic material to obtain a weight gain level from about 2 to about 30 percent, although the overcoat may be greater depending upon the physical properties of the particular analgesic drug compound utilized and the desired release rate, among other things. In such a case, the nontoxic NMDA receptor antagonist may be applied to the exterior surface of, or included within, the hydrophobic coating to provide for the immediate release of the nontoxic NMDA receptor antagonist while at the same time providing for the extended release of the analgesic drug.

The solvent which is used for the hydrophobic material may be any pharmaceutically acceptable solvent, including water, methanol, ethanol, methylene chloride and mixtures thereof. It is preferable however, that the coatings be based upon aqueous dispersions of the hydrophobic material.

In certain preferred embodiments of the present invention, the hydrophobic polymer comprising the sustained-release coating is a pharmaceutically acceptable acrylic polymer, including but not limited to acrylic acid and methacrylic acid copolymers, methacrylic acid copolymers, methyl methacrylate copolymers, ethoxyethyl methacrylates, cynaoethyl methacrylate, methyl methacrylate, copolymers, methacrylic acid copolymers, methyl methacrylate copolymers, methyl methacrylate copolymers, methyl methacrylate copolymers, methacrylic acid copolymer, aminoalkyl methacrylate copolymer, methacrylic acid copolymers, methyl methacrylate copolymers, poly(acrylic acid), poly(methacrylic acid, methacrylic acid alkylamide copolymer, poly(methyl methacrylate), poly(methacrylic acid)(anhydride), methyl methacrylate, polymethacrylate, methyl methacrylate copolymer, poly(methyl methacrylate), poly(methyl methacrylate)copolymer, polyacrylamide, aminoalkyl methacrylate copolymer, poly(methacrylic acid anhydride), and glycidyl methacrylate copolymers.

In other preferred embodiments, the hydrophobic polymer which may be used for coating the substrates of the present invention is a hydrophobic cellulosic material such as ethylcellulose. Those skilled in the art will appreciate that other cellulosic polymers, including other alkyl cellulosic polymers, may be substituted for part or all of the ethylcellulose included in the hydrophobic polymer coatings of the present invention.

In embodiments of the present invention where the coating comprises an aqueous dispersion of a hydrophobic polymer, the inclusion of an effective amount of a plasticizer in the aqueous dispersion of hydrophobic polymer will further improve the physical properties of the film. For example, because ethylcellulose has a relatively high glass transition temperature and does not form flexible films under normal coating conditions, it is necessary to plasticize the ethylcellulose before using the same as a coating material. Generally, the amount of plasticizer included in a coating solution is based on the concentration of the film-former, e.g., most often from about 1 to about 50 percent by weight of the film-former. Concentration of the plasticizer, however, can only be properly determined after careful experimentation with the particular coating solution and method of application.

Examples of suitable plasticizers for ethylcellulose include water insoluble plasticizers such as dibutyl sebacate, diethyl phthalate, triethyl citrate, tributyl citrate, and triacetin, although it is possible that other water-insoluble plasticizers (such as acetylated monoglycerides, phthalate esters, castor oil, etc.) may be used. Triethyl citrate is especially preferred.

Examples of suitable plasticizers for the acrylic polymers of the present invention include citric acid esters such as triethyl citrate NF XVI, tributyl citrate, dibutyl phthalate, and possibly 1,2-propylene glycol polyethylene glycols, propylene glycol, diethyl phthalate, castor oil, and triacetin, although it is possible that other water-insoluble plasticizers (such as acetylated monoglycerides, phthalate esters, castor oil, etc.) may be used. Triethyl citrate is especially preferred.

The sustained-release profile of the formulations of the invention can be altered, for example, by varying the thickness of the hydrophobic coating, changing the particular hydrophobic material used, or altering the relative amounts of, e.g., different acrylic resin lacquers, altering the manner in which the plasticizer is added (e.g., when the sustained-release coating is derived from an aqueous dispersion of hydrophobic polymer), by varying the amount of plasticizer relative to hydrophobic polymer, by the inclusion of additional ingredients or excipients, by altering the method of manufacture, etc. As noted above, the nontoxic NMDA receptor antagonist may be applied to the exterior of, or contained within, any coating of a carrier containing an analgesic drug to provide for the immediate release of the nontoxic NMDA receptor antagonist while at the same time providing for the extended release of the analgesic drug.

Sustained-release spheroids or beads, coated with a therapeutically active agent are prepared, e.g. by dissolving the analgesic drug in water and then spraying the solution onto a substrate using a Wurster insert. Optionally, additional ingredients are also added prior to coating the beads in order to assist the analgesic drug binding to the substrates, and/or to color the solution, etc. For example, a product which includes hydroxypropyl methylcellulose, etc. with or without colorant may be added to the solution and the solution mixed (e.g., for about 1 hour) prior to application of the same onto the beads. The resultant coated substrate, in this example beads, may then be optionally overcoated with a barrier agent, to separate the therapeutically active agent from the hydrophobic sustained-release coating. An example of a suitable barrier agent is one which comprises hydroxypropyl methylcellulose. However, any film-former known in the art maybe used. It is preferred that the barrier agent does not affect the dissolution rate of the final product.

The coating solutions of the present invention may contain, in addition to the film-former, plasticizer, and solvent system (i.e., water), a colorant to provide elegance and product distinction. Color may be added to the solution of the therapeutically active agent instead, or in addition to the aqueous dispersion of hydrophobic polymer.

The plasticized aqueous dispersion of hydrophobic polymer may be applied onto the substrate comprising the therapeutically active agent, i.e., analgesic drug, by spraying using any suitable spray equipment known in the art. In a preferred method, a Wurster fluidized-bed system is used in which an air jet, injected from underneath, fluidizes the core material and effects drying while the acrylic polymer coating is sprayed on. A sufficient amount of the aqueous dispersion of hydrophobic polymer to obtain a predetermined sustained-release of said therapeutically active agent when said coated substrate is exposed to aqueous solutions, e.g. gastric fluid, is preferably applied, taking into account the physically characteristics of the therapeutically active agent, the manner of incorporation of the plasticizer, etc. After coating with the hydrophobic polymer, a further overcoat of a film-former is optionally applied to the beads. This overcoat is provided, if at all, in order to substantially reduce agglomeration of the beads.

Next, the coated beads are cured in order to obtain a stabilized release rate of the therapeutically active agent.

In another embodiment, the analgesic composition of the present invention is an aqueous suspension. Aqueous suspensions can contain the analgesic drug and nontoxic NMDA receptor antagonist in admixture with pharmaceutically acceptable excipients such as suspending agents, e.g., sodium carboxymethyl cellulose, methylcellulose, hydroxypropylmethylcellulose, sodium alginate, polyvinylpyrrolidone, and natural gums such as gum tragacanth and gum acacia; dispersing or wetting agents such as naturally occurring phosphatide and lecithin, or condensation products of an alkylene oxide with fatty acids, e.g., polyoxyethylene stearate, or condensation products of ethylene oxide with long chain aliphatic alcohols, e.g., heptadecaethyleneoxycetanol, or condensation products of ethylene oxide with partial esters derived from fatty acids and a hexitol, e.g., polyoxyethylene sorbitol monoleate or condensation products of ethylene oxide with partial esters derived from fatty acids and hexitol anhydrides, e.g., polyoxyethylene sorbitan monooleate. Such aqueous suspensions can also contain one or more preservatives, e.g., ethyl- or n-propyl-p-hydroxy benzoate, one or more coloring agents, one or more flavoring agents and one or more sweetening agents, such as sucrose, saccharin or sodium or calcium cyclamate. In such an aqueous suspension, the analgesic drug is in an extended release form and the nontoxic NMDA receptor antagonist is in an immediate release form.

Dispersible powders and granules suitable for preparation of an aqueous suspension by the addition of water provide the composition in admixture with a dispersing of wetting agent, suspending agents and one or more preservatives. Suitable dispersing or wetting agents and suspending agents are exemplified by those already mentioned above. Additional excipients, e.g., sweetening, flavoring and coloring agents, can also be present. Syrups and elixirs can be formulated with sweetening agents, for example glycerol, sorbitol or sucrose. Such formulations can also contain a demulcent, a preservative and flavoring and coloring agents.

The analgesic composition herein can be formulated as a solid, liquid, powder, elixir, injectable solution, etc. When formulated for oral delivery, the combination of drugs herein may be in the form of tablets, liquids, troches, lozenges, quick dissolve tablets, aqueous or oily suspensions, multiparticulate formulations including dispersible powders, granules, carrier spheroids or coated inert beads, emulsions, hard or soft capsules or syrups or elixirs, microparticles (e.g., microcapsules, microspheres and the like), buccal tablets, etc. The analgesic drug and nontoxic NMDA receptor antagonist can be employed in admixtures with conventional excipients, i.e., pharmaceutically acceptable organic or inorganic substances suitable for oral administration, known to those skilled in the art. Suitable pharmaceutically acceptable substances include but are not limited to water, salt solutions, alcohols, gum arabic, vegetable oils, benzyl alcohols, polyethylene glycols, gelate, carbohydrates such as lactose, amylose or starch, magnesium stearate, talc, silicic acid, viscous paraffin, perfume oil, fatty acid monoglycerides and diglycerides, pentaerythritol fatty acid esters, hydroxymethylcellulose, polyvinylpyrrolidone, etc. The pharmaceutical preparations can be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They can also be combined where desired with other active agents, e.g., other analgesic agents. For oral administration, particularly suitable are tablets, dragees, liquids, drops, suppositories, or capsules, caplets and gelcaps. The compositions intended for oral use may be prepared according to any method known in the art. When prepared as tablets, the tablets may be uncoated or they may be coated by known techniques for elegance or to further delay release of the active ingredients. Formulations for oral use may also be presented as hard gelatin capsules wherein the active ingredient is mixed with an inert diluent.

It will be understood that various modifications may be made to the embodiments disclosed herein. Therefore, the above description should not be construed as limiting, but merely as exemplifications of preferred embodiments. For example, NMDA receptor antagonists other than dextromethorphan can be utilized in the analgesic composition described herein. Those skilled in the art will envision other modifications within the scope and spirit of the claims appended hereto.

1-49. (canceled)

50. An analgesic composition which comprises at least one analgesic drug in an extended release form in combination with an analgesic-enhancing amount of at least one non-toxic N-methyl-D-aspartate receptor antagonist in an immediate release form.

51. The analgesic composition of claim 50, wherein
the nontoxic NMDA receptor antagonist is at least one member selected from the group consisting of dextromethorphan, dextrorphan, memantine, amantidine, d-methadone and their pharmaceutically acceptable salts; or
the nontoxic NMDA receptor antagonist is present in an immediate release carrier; or
the analgesic drug is selected from the group consisting essentially of non-narcotic analgesics, coal tar analgesics, nonsteroidal anti-inflammatory drugs, gabapentin, substance P antagonists, capsaicin, capsaicinoids, and cyclooxygenase-II (COX II) inhibitors; or
the weight ratio of the analgesic drug to the nontoxic NMDA receptor antagonist ranges from about 2:1 to about 1:10; or
the weight ratio of the analgesic drug to the nontoxic NMDA receptor antagonist ranges from about 1:1 to about 1:5.

52. The analgesic composition of claim 50, wherein the analgesic drug is an analgesically effective amount of at least one opioid analgesic and the analgesic composition is substantially free of opioid antagonist.

53. The analgesic composition of claim 53 wherein the opioid analgesic is at least one member selected from the group consisting of alfentanil, allylprodine, alphaprodine, anileridine, benzylmorphine, bezitramide, buprenorphine, butorphanol, clonitazine, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethymethylthiambutene, ethylmorphine, etonitazene, fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphine, normorphine, norpipanone, opium, oxycodone, oxymorphone, papaveretum, pentazocine, phenadoxne, phenomorphan, phenazocine, phenoperidine, piminodine, piritramide, propheptazine, promedol, properidine, propoxyphene, sufentanyl, tilidine, tramadol and their pharmaceutically acceptable salts.

54. The analgesic composition of claim 53 wherein the opioid analgesic is at least one member selected from the group consisting of codeine, dihydrocodeine, hydrocodone, hydromorphone, levorphanol, meperidine, methadone, morphine, oxycodone, oxymorphone, propoxyphene and their pharmaceutically acceptable salts.

55. The analgesic composition of claim 53 wherein the nontoxic NMDA receptor antagonist is at least one member selected from the group consisting of dextromethorphan, dextrorphan, memantine, amantidine, d-methadone and their pharmaceutically acceptable salts.

56. The analgesic composition of claim 50, wherein the extended release form is an extended release carrier comprising a base material selected from the group consisting of a hydrophilic polymer, a hydrophobic polymer, a long chain hydrocarbon, a polyalkylene glycol, higher aliphatic alcohols, acrylic resins, and mixtures thereof.

57. The analgesic composition of claim 56, wherein the nontoxic NMDA receptor antagonist is applied to the extended release carrier's exterior surface.

58. The analgesic composition of claim 50, wherein the extended release form comprises a base material having a coating that controls the release of the analgesic drug.

59. The analgesic composition of claim 58, wherein the coating includes the nontoxic NMDA receptor antagonist.

60. The analgesic composition of claim 50, which is a liquid dosage form.

61. The analgesic composition of claim 60, which is an injectable dosage form.

62. The analgesic composition of claim 52, wherein
the weight ratio of the opioid analgesic to the nontoxic NMDA receptor antagonist is about 1:1; or
the daily dosage of opioid analgesic is from about 1 mg to about 800 mg per 70 kg body weight and the daily dosage of nontoxic NMDA receptor antagonist is from about 10 mg to about 750 mg per 70 kg body weight; or
the daily dosage of opioid analgesic is from about 10 mg to about 500 mg per 70 kg body weight and the daily dosage of nontoxic NMDA receptor antagonist is from about 30 mg to about 500 mg per 70 kg body weight; or
the opioid analgesic is selected from the group consisting of fentanyl and sufentanyl in a daily dosage of from about 100 μg to about 6 mg per 70 kg body weight and the daily dosage of nontoxic NMDA receptor antagonist is from about 10 mg to about 750 mg per 70 kg body weight.

63. An analgesic composition which comprises an analgesic effective amount of at least one opioid analgesic selected from the group consisting of codeine, dihydrocodeine, hydrocodone, hydromorphone, levorphanol, meperidine, methadone, morphine, oxycodone, oxymorphone, propoxyphene, tramadol and their pharmaceutically acceptable salts in an extended release form, and an opioid analgesia-enhancing amount of dextromethorphan in an immediate release form, wherein the analgesic composition is substantially free of opioid antagonist.

64. The analgesic composition of claim 63 wherein
the dextromethorphan is present in an immediate release carrier;
the weight ratio of the opioid analgesic to the nontoxic NMDA receptor antagonist ranges from about 2:1 to about 1:10; or
the weight ratio of the opioid analgesic to the nontoxic NMDA receptor antagonist ranges from about 1:1 to about 1:5; or
the weight ratio of the opioid analgesic to the dextromethorphan is about 1:1; or
the daily dosage of opioid analgesic is from about 1 mg to about 800 mg per 70 kg body weight and the daily dosage of dextromethorphan is from about 10 mg to about 750 mg per 70 kg body weight; or
the daily dosage of opioid analgesic is from about 10 mg to about 500 mg per 70 kg body weight and the daily dosage of dextromethorphan is from about 30 mg to about 500 mg per 70 kg body weight per unit dose.

65. The analgesic composition of claim 63 wherein the extended release form is an extended release carrier comprising a base material selected from the group consisting of hydrophilic polymer, a hydrophobic polymer, a long chain hydrocarbon, a polyalkylene glycol, higher aliphatic alcohols, acrylic resins, and mixtures thereof.

66. The analgesic composition of claim 65 wherein the dextromethorphan is applied to the extended release carrier's exterior surface.

67. An analgesic composition consisting essentially of at least one analgesic drug in an extended release form in combination with an analgesia-enhancing amount of at least one nontoxic N-methyl-D-aspartate receptor antagonist in an immediate release form.

68. The analgesic composition of claim 67 wherein
the nontoxic NMDA receptor antagonist is at least one member selected from the group consisting of dextromethorphan, dextrorphan, memantine, amantidine, d-methadone, and their pharmaceutically acceptable salts; or
the nontoxic NMDA receptor antagonist is present in an immediate release carrier; or
the analgesic drug is selected from the group consisting essentially of non-narcotic analgesic, coal tar analgesics, nonsteroidal anti-inflammatory drugs, gabapentin, substance P antagonists, capsaicin, capsaicinoids, and cyclooxygenase-II (COX II) inhibitors; or
the weight ratio of the analgesic drug to the nontoxic NMDA receptor antagonist ranges from about 2:1 to about 1:10; or
the weight ratio of the analgesic drug to the nontoxic NMDA receptor antagonist ranges from about 1:1 to about 1:5; or
the weight ratio of the analgesic drug to the nontoxic NMDA receptor antagonist is about 1:1.

69. The analgesic composition of claim 67 wherein the analgesic drug is an analgesically effective amount of at least one opioid analgesic and the analgesic composition is substantially free of opioid antagonist.

70. The analgesic composition of claim 69 wherein the opioid analgesic is at least one member selected from the group consisting of alfentanil, allylprodine, alphaprodine, anileridine, benzylmorphine, bezitramide, buprenorphine, butorphanol, clonitazine, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethymethylthiambutene, ethylmorphine, etonitazene, fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, naceine, nicomorphine, norlevorphanol, normethadone, nalorphine, nalbuphine, normorphine, norpipanone, opium, oxycodone, oxymorphone, papaveretum, pentazocine, phenadoxne, phenomorphan, phenazocine, phenoperidine, piminodine, piritramide, propheptazine, promedol, properidine, propoxyphene, sufentanyl, tilidine, tramadol and their pharmaceutically acceptable salts; or
the opioid analgesic is at least one member selected from the group consisting of codeine, dihydrocodeine, hydrocodone, hydromorphone, levorphanol, meperidine, methadone, morphine, oxycodone, oxymorphone, propoxyphene and their pharmaceutically acceptable salts; or
the nontoxic NMDA receptor antagonist is at least one member selected from the group consisting of dextromethorphan, dextrorphan, memantine, amantidine, d-methadone and their pharmaceutically acceptable salts; or
the weight ratio of the opioid analgesic to the nontoxic NMDA receptor antagonist ranges from about 2:1 to about 1:10; or
the weight ratio of the opioid analgesic to the nontoxic NMDA receptor antagonist ranges from about 1:1 to about 1:5; or
the weight ratio of the opioid analgesic to the nontoxic NMDA receptor antagonist is about 1:1.

71. The analgesic composition of claim 67 wherein the extended release form is an extended release carrier comprising a base material selected from the group consisting of a hydrophilic polymer, a hydrophobic polymer, a long chain hydrocarbon, a polyalkylene glycol, higher aliphatic alcohols, acrylic resins, and mixtures thereof.

72. The analgesic composition of claim 71 wherein the nontoxic NMDA receptor antagonist is applied to the extended release carrier's exterior surface.

73. The analgesic composition of claim 67 wherein the extended release form comprises a base material having a coating that controls the release of the analgesic drug.

74. The analgesic composition of claim 73 wherein the coating includes the nontoxic NMDA receptor antagonist.

75. The analgesic composition of claim 67 which is a liquid dosage form.

76. The analgesic composition of claim 75 which is an injectable dosage form.
